(19) United States
(12) Patent Application Publication
(10) Pub. No.: US 2022/0068485 A1
(43) Pub. Date: Mar. 3, 2022

(54) COMPUTING SYSTEM THAT GENERATES PATIENT-SPECIFIC OUTCOME PREDICTIONS

(71) Applicant: Right Mechanics, Inc., Westlake Village, CA (US)

(72) Inventors: Anish Potty, Laredo, TX (US); Ajish Potty, Missouri City, TX (US); Rithesh Punyamurthula, Simi Valley, CA (US)

(21) Appl. No.: 17/003,459

(22) Filed: Aug. 26, 2020

(51) Int. Cl. G16H 50/30; G16H 50/20; G16H 10/20; G16H 10/60

(52) U.S. Cl. CPC G16H 50/30; G16H 10/60; G16H 10/20; G16H 50/20

(57) ABSTRACT

A computing system receives clinical data for a patient that is to undergo treatment for a medical problem and a pre-treatment score value that is indicative of a condition of the patient prior to undergoing the treatment. The computing system provides values in the clinical data and the pre-treatment score value as input to a computer-implemented model. The computer-implemented model outputs, based upon the input, a predicted difference value that is indicative of a predicted change in the condition of the patient from a first point in time occurring prior to the patient undergoing the treatment to a second point in time occurring subsequent to the patient undergoing the treatment. The computing system generates a predicted post-treatment score value for the patient based upon the pre-treatment score value and the predicted difference value, and causes the predicted post-treatment score value to be presented on a display.

### BACKGROUND

Medical software is often used by healthcare workers to generate outcome measures (i.e., scores) that evaluate the efficacy of treatment of medical problems of patients. For instance, conventional medical software may receive a score input by a physician for a treatment for a certain musculoskeletal problem (e.g., knee, hip, spine, and shoulder disorders) of a patient, wherein the score is indicative of a condition of the patient at the time the score is generated. In practice, outcome measures tend to be based upon physician derived objective evaluations input to the medical software, such as a range of motion evaluation or a radiographic report. However, objective evaluations alone tend to have minimal input from a patient, which tend to limit their effectiveness in properly assessing the condition of the patient.

As a result, patient-reported outcome measures (PROMs) have been developed that utilize subjective experiences of a patient (e.g., pain level) in addition to objective evaluations in assessing a condition of the patient. Examples of PROMs include the American Shoulder and Elbow Surgeons Score (ASES) and the Knee Injury and Osteoarthritis Outcome Score (KOOS). For a typical PROM, medical software presents a questionnaire on a display, wherein the questionnaire comprises questions that pertain to subjective and objective experiences of the patient with respective to the medical problem. The patient reads the questions in the questionnaire (or a healthcare worker treating the patient reads the questions to the patient) and provides answers to the questions. The medical software receives the answers to the questions and generates a PROM score that is indicative of the condition of the patient. In an example, the PROM score may range from 0 to 100, with a 0 being indicative of a relatively poor condition of the patient and with a 100 being indicative of a relatively healthy condition of the patient. Typically, the PROM score is relatively low prior to the patient undergoing treatment for the medical problem and relatively high after the patient has undergone the treatment for the medical problem. The medical software may generate PROM scores at different points in time (e.g., before treatment, three months after treatment, six months after treatment, and so forth) based upon respective answers to the questions in the questionnaire at the different points in time to enable the healthcare worker to evaluate how the patient is responding to the treatment.

Some conventional medical applications may be configured to execute a machine learning model (e.g., a classification model) in order to predict patient outcomes. A healthcare worker and/or a patient can utilize a predicted patient outcome to make decisions (e.g., changes to treatment procedures, changes in patient behavior, etc.) that will likely lead to faster recovery. However, as most patients tend to respond well to treatment (i.e., the treatment alleviates the medical problem) as more time elapses from a time at which the treatment begins, PROM scores for patients who undergo the treatment tend to be clustered at a higher end of a scale for the PROM scores, particularly as the patients are farther removed from the treatment (e.g., twelve months after the treatment). This leads to the PROM scores having a skewed distribution. Machine learning models trained upon a skewed distribution of data tend to generate inaccurate predictions when executed. Thus, conventional medical applications that execute machine learning models tend to be limited in their ability to accurately forecast patient outcomes when undergoing a treatment for a medical problem.

### SUMMARY

The following is a brief summary of subject matter that is described in greater detail herein. This summary is not intended to be limiting as to the scope of the claims.

Described herein are various technologies pertaining to generating patient-specific outcome predictions. With more particularity, a computing system is described herein that is configured to train, execute, and update a computer-implemented model. The computing system, by executing the computer-implemented model, outputs a predicted post-treatment score value for a treatment for a medical problem of patient. A healthcare worker may utilize the predicted post-treatment score value to evaluate whether the patient is responding properly to the treatment, and to tailor further treatment thereto.

In operation, a computing system accesses training data comprising values for features associated with a plurality of patients who have undergone treatment for a medical problem. In an example, the treatment may be shoulder arthroscopic surgery and the medical problem may be a shoulder tear. The features include demographic information, factors pertaining to the medical problem, a pre-treatment score, and a post-treatment score (or several post-treatment scores that are generated at different times after each of the plurality of patients have undergone the treatment). Likewise, the values for the features include demographic information values, values for the factors pertaining to the medical problem, a pre-treatment score value, and a post-treatment score value. In an example, a first feature in the features may be patient age, and the value for the patient age feature may be “50.” In another example, a second feature in the features may be a tear shape feature, and a value for the tear shape may be “radial.”

Pre-treatment score values and post-treatment score values are indicative of conditions of the plurality of patients prior to and subsequent to undergoing the treatment, respectively. The pre-treatment score values and the post-treatment score values have been generated based upon answers to questions in a questionnaire, the answers having been provided by each of the plurality of patients. In an example, the pre-treatment score and the post-treatment score may be a patient-report outcome measure (PROM), such as an American Shoulder and Elbow Surgeons Score (ASES). The post-treatment score values for the plurality of patients tend to have an (approximately) skewed distribution. The computing system subtracts each of the pre-treatment score values from each of the post-treatment score values to generate difference values for the plurality of patients, each difference value in the difference values corresponding to a different patient in the plurality of patients, thus generating a difference feature in the training data. Unlike the post-treatment score values alone, the difference values have an (approximately) normal distribution. The computing system trains a computer-implemented model based upon the difference feature and at least one other feature in the features, wherein a target variable of the computer-implemented model is the difference feature. In an example, the computer-implemented model may be a regression model, such as gradient boosted decision tree regression model. The computing system may also test the computer-implemented model on test data.

Subsequently, the computing system receives clinical data for a patient that is to undergo the treatment for the medical problem and a pre-treatment score value for the patient that is indicative of a condition of the patient prior to the patient undergoing the treatment. The pre-treatment score value for the patient has been generated based upon answers to the questions in the questionnaire that pertain to the medical problem, the answers having been provided by the patient. The computing system provides values in the clinical data and the pre-treatment score value to the computer-implemented model as input. The computing system executes the computer-implemented model, and the computer-implemented model outputs, based upon the input, a predicted difference value that is indicative of a predicted change in the condition of the patient from a first point in time to a second point in time (e.g., three months after undergoing the treatment, six months after undergoing the treatment, etc.). The computing system generates a predicted post-treatment score value based upon the predicted difference value and the pre-treatment score value, and causes the predicted post-treatment score value (or an indication thereof) to be presented on a display.

In an example, the computer-implemented model can output several predicted post-treatment score values. For instance, the computer-implemented model can output a first predicted post-treatment score value corresponding to three months after the patient undergoes the treatment, a second predicted post-treatment score value corresponding to six months after the patient undergoes the treatment, and a third predicted post-treatment score value corresponding to twelve months after the patient undergoes the treatment. The computing system can cause the pre-treatment score value, the first predicted post-treatment score value, the second predicted post-treatment score value, and the third predicted post-treatment score value to be presented in a plot of scores versus time shown on a display in order to enable a healthcare worker to visualize a predicted recovery of the patient. In an example, the patient may be visiting the healthcare worker for a three month check-up appointment after undergoing the treatment for the medical problem. The computing device operated by the healthcare worker can generate a post-treatment score value (for instance, by having the patient answer questions in a questionnaire, such as an ASES questionnaire) for the patient and display the post-treatment score value on the plot along with the first predicted post-treatment score value referenced above. The healthcare worker may then tailor further treatment for the patient based upon a comparison between the post-treatment score value and the first predicted post-treatment score value. For instance, if the post-treatment score value is less than the first predicted post-treatment score value, the patient may require additional treatments to address the medical problem. The computing device may also transmit the post-treatment score value to the computing system, and the computing system may update (e.g., retrain) the computer-implemented model based upon the post-treatment score value.

If there are multiple treatments available for the medical problem, the computing system may generate different computer-implemented models for each of the treatments and/or the computing system may optimize the computer-implemented model by executing the model using a large range of inputs or by executing optimization algorithms such as a genetic algorithm, an exhaustive search, Bayesian optimization, etc. For instance, for the medical problem that the patient is experiencing, there may be a first treatment (e.g., a first surgical implant, a first type of surgery, a first medication, a first surgeon that performs a surgery, etc.) and a second treatment (e.g., a second surgical implant, a second type of surgery, a second medication, a second surgeon that performs the surgery, etc.) available. As such, the training data may comprise a first subset corresponding to patients that underwent the first treatment for the medical problem and a second subset corresponding to patients that underwent the second treatment for the medical problem. Using the above-described processes, the computing system may generate a first computer-implemented model assigned to the first treatment and a second computer-implemented model assigned to the second treatment based upon the first subset of training data and the second subset of training data, respectively. The first computer-implemented model and the second computer-implemented model may output a first predicted post-treatment score value for the first treatment and a second predicted post-treatment score value for the second treatment based upon values in clinical data of the patient and the pre-treatment score value for the patient, and the first predicted post-treatment score value and the second predicted post-treatment score value may be presented on a display to the healthcare worker. The healthcare worker may decide which treatment to utilize based upon a comparison between the first predicted post-treatment score value and the second predicted post-treatment score value. Alternatively, factors pertaining to each of the multiple treatments may be used as features of the computer-implemented model.

The above-described technologies present various advantages over conventional computer-implemented approaches for forecasting patient recovery. First, unlike conventional computer-implemented approaches for forecasting patient recovery that tend to utilize classification models, the above-described technologies employ a regression model, which provides a more finely detailed measure of patient recovery. Second, by utilizing a difference between a post-treatment score and a pre-treatment score for the treatment (or, more generally a difference between a post-treatment score and a most recently reported score for the treatment), the difference being normally distributed, as a target variable for the computer-implemented model, the model generates more accurate predictions than conventional approaches. Third, by generating computer-implemented models for different treatments for a medical problem, and outputting predicted post-treatment score values for each of the treatments, the above-described technologies enable a healthcare worker to better select a treatment for the medical problem.

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### BRIEF DESCRIPTION OF THE DRAWINGS

FIG. 1 is a functional block diagram of an exemplary computing system that facilitates generating patient-specific outcome predictions.

FIG. 2 is a functional block diagram of another exemplary computing system that facilitates generating patient-specific outcome predictions.

FIG. 3 is a plot of score versus time.

FIG. 4 is a flow diagram that illustrates an exemplary methodology performed by a computing system for training a computer-implemented model that generates patient-specific outcome predictions.

FIG. 5 is a flow diagram that illustrates an exemplary methodology performed by a computing system for executing a computer-implemented model that generates patient-specific outcome predictions.

FIG. 6 is a flow diagram that illustrates an exemplary methodology performed by a client computing device for displaying patient-specific outcome predictions.

FIG. 7 illustrates several histograms of American Shoulder and Elbow Surgeons Scores (ASESs).

FIG. 8 illustrates several parity plots of observed and predicted changes in ASESs.

FIG. 9 is an exemplary computing device.

### DETAILED DESCRIPTION

Various technologies pertaining to generating patient-specific outcome predictions are now described with reference to the drawings, wherein like reference numerals are used to refer to like elements throughout. In the following description, for purposes of explanation, numerous specific details are set forth in order to provide a thorough understanding of one or more aspects. It may be evident, however, that such aspect(s) may be practiced without these specific details. In other instances, well-known structures and devices are shown in block diagram form in order to facilitate describing one or more aspects. Further, it is to be understood that functionality that is described as being carried out by certain system components may be performed by multiple components. Similarly, for instance, a component may be configured to perform functionality that is described as being carried out by multiple components.

Moreover, the term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

Further, as used herein, the terms “component,” “application,” “module,” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices. Further, as used herein, the term “exemplary” is intended to mean serving as an illustration or example of something, and is not intended to indicate a preference.

Additionally, as used herein, the term “medical problem” is meant to encompass a health condition of the patient that affects the well-being of a patient. For instance, medical problems may include injuries, diseases, and/or allergies. As used herein, the term “treatment” is meant to encompass a course of action that attempts to alleviate a medical problem. For instance, treatments for medical problems may include surgical procedures, taking medications, undergoing non-surgical therapies, or any combination thereof.

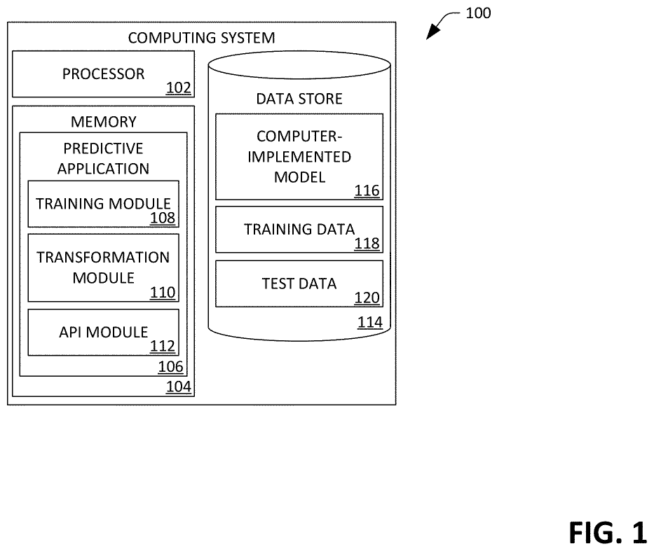

With reference to FIG. 1, an exemplary computing system 100 that facilitates generating patient-specific outcome predictions is illustrated. In an embodiment, the computing system 100 may be a cloud-based computing platform. The computing system 100 includes a processor 102 and memory 104, wherein the memory 104 has a predictive application 106 loaded therein. As will be described in greater detail below, the predictive application 106, when executed by the processor 102, is generally configured to generate a predicted post-treatment score value for a patient, the predicted post-treatment score value being a forecast as to how a patient will respond to a treatment for a medical problem. In an embodiment, the predictive application 106 may be a module of an electronic health records (EHR) application stored in the memory 104.

The predictive application 106 comprises a training module 108. The training module 108 is generally configured to train computer-implemented models based upon training data. As such, the training module 108 may be configured to select some or all of the features in the training data upon which the computer-implemented models are to be trained. The training module 108 is further configured to test performance of the computer-implemented models based upon test data. Additionally, the training module 108 is further configured to update (e.g., retrain) the computer-implemented models based upon actual performance of the computer-implemented models.

The predictive application 106 further comprises a transformation module 110. The transformation module 110 is generally configured to perform transformations on values for features that are to be used to train computer-implemented models and to perform transformations on input values for features that are to be input to the computer-implemented models. For instance, transformations may include converting non-numerical values to numerical values, converting data types of the values (e.g., integer to float), and so forth. The transformations may also include generating new features (and corresponding values) by applying mathematical operations (e.g., addition, subtraction, multiplication, division, etc.) on values for features in the training data.

The predictive application 106 also comprises an application programming interface (API) module 112. The API module 112 is generally configured to expose an API to computing devices. To this end, the API module 112 is configured to receive an API call from a computing device, and to provide the computing device with a predicted post-treatment score value for a treatment for a patient responsive to receiving the API call (described in greater detail below).

The computing system 100 further includes a data store 114. The data store 114 stores a computer-implemented model 116 that is generated by the training module 108 of the predictive application 106. The computer-implemented model 116 is a regression model. In an embodiment, the computer-implemented model 116 may be one or more of a linear regression, lasso, ridge regression, a decision tree, a gradient boosted decision tree, a random forest, an artificial neutral network (ANN), a support vector machine (SVM), a hidden Markov model, a recurrent neural network (RNN), a deep neural network (DNN), a convolutional neural network (CNN), etc. In an embodiment, the computer-implemented model 116 may be a computer-implemented machine learning model.

The data store 114 also stores training data 118 that the predictive application 106 uses to train the computer-implemented model 116. The training data 118 comprises values for features associated with a plurality of patients that have undergone a treatment for a medical problem. The training data 118 may be organized into sets, wherein each set in the sets comprises data pertaining to a different patient. In general, the features of the training data 118 include demographic information and factors pertaining to the medical problem. The demographic information may include gender, age, body mass index (BMI), whether or not a patient smokes, and/or presence or absence of diabetes. In a specific example where the treatment undergone by each of the plurality of patients is shoulder arthroscopic surgery, the factors pertaining to the medical problem include tendons torn, tendon quality, cofield tear size, retraction stage, tear shape, medial anchor type, medial knotless anchors, medial suture anchors, lateral anchor type, lateral knotless anchors, lateral suture anchors, pre-treatment visual analog scale pain score (PT VAPS), pre-treatment American Shoulder and Elbow Surgeons score (PT ASES), post-surgery icing protocol, type of brace, types of physiotherapy exercises, duration and frequency of exercise, how closely the patient followed doctor recommended protocols, and/or a date at which treatment began. Values for the features may be collected by a human, a software interface, and/or a virtual conversational assistant. The virtual conversational assistant, based upon the discussion with the patient, may determine relevant PROMs to collect from the patient. The computer-implemented model 116 may change its recommendations based on the values for the features (e.g., changes in brace type, changes in physiotherapy protocol that would result in fastest recovery, etc.) Each feature in the features has a corresponding value for each patient. For instance, a first feature in the features may be BMI and hence a value for the first feature for a first patient in the plurality of patients may be a numerical value, such as “23.5,” and a value for the first feature for a second patient in the plurality of patients may be “30.2.” In another example, a second feature in the features may be tear shape, and hence a value for the second feature for the first patient may be an identifier for the tear shape, such as “radial,” and a value for the second feature for the second patient may be “U-shaped.” Thus, it is to be understood that the values for the features in the training data 118 may be numerical or non-numerical (e.g., a string of alphanumeric characters, boolean, etc.).

The features of the training data 118 also include a pre-treatment score and at least one post-treatment score, as well as corresponding values for the pretreatment score and the at least one post-treatment score. In general, the pre-treatment score and the at least one post-treatment score relate to a condition of a patient before and after treatment for a medical problem, respectively. In an embodiment, a scale of the scores may range from 0 to 100. Relatively higher scores may indicate that a patient that feels relatively “good”, while relatively lower scores may indicate that the patient feels relatively “bad.” In an example, the at least one post-treatment score may comprise a three month post-treatment score, a six month post-treatment score, and a twelve month post-treatment score. A pre-treatment score value and at least one post-treatment score value may be generated by a computing device based upon answers to questions in a questionnaire, the answers being provided by the patient. In an embodiment, the pre-treatment score and the post-treatment score may be a patient-reported outcome measures (PROMs).

In an embodiment, the pre-treatment score may be a pre-treatment American Shoulder and Elbow Surgeons Score (ASES) and the at least one post-treatment score may be a post-treatment ASES score. The ASES is a mixed outcome reporting measure, consisting of both a physician-rated component and a patient-reported component. The ASES is applicable for use in all patients with shoulder pathologies independent of specific diagnosis of each of the patients. The ASES is validated for several conditions, including osteoarthritis, shoulder instability, rotator-cuff injuries, and shoulder arthroplasty.

In another embodiment, the pre-treatment score may be a pre-treatment Knee Injury and Osteoarthritis Score (KOOS) and the at least one post-treatment score may be a post-treatment KOOS. The KOOS is generated based upon answers to a questionnaire that is designed to assess short and long-term patient-relevant outcomes following knee injury. The KOOS is self-administered by a patient and assess five outcomes: pain, symptoms, activities of daily living, sport and recreation function, and knee-related quality of life.

The data store 114 also stores test data 120 that the predictive application 106 uses to test the computer-implemented model 116 after the predictive application 106 has trained the computer-implemented model 116 using the training data 118. The test data 120 comprises the features (described above) of the training data 118, but corresponds to patients that are different than the plurality of patients and hence the test data 120 has different values for the features than those of the training data 118. In an embodiment, the training data 118 and the test data 120 may be organized within a computer-implemented database stored within the data store 114, such as a Structured Query Language (SQL) database. In an embodiment, the training data 118 and the test data 120 are de-identified to preserve patient anonymity. In an embodiment, for the combined training data 118 and the test data 120, the training data 118 may comprise 80% of available data and the test data 120 may comprise 20% of the available data. However, other possibilities are contemplated.

Operation of the computing system 100 is now set forth. The predictive application 106 accesses the training data 118. As noted above, the training data 118 comprises post-treatment score values and pre-treatment score values for the plurality of patients. The post-treatment score values have an (approximately) skewed distribution. The predictive application 106 subtracts each of the pre-treatment score values from each of the post-treatment score values to generate difference values for the plurality of patients, each difference value in the difference values corresponding to a different patient in the plurality of patients. The predictive application 106 may store the difference values as part of the training data 118. For instance, for a set in the training data 118 of a patient, the predictive application may subtract a pre-treatment score value for the patient from a post-treatment score value for the patient to generate a difference value. The difference values generated by the predictive application 106 have an (approximately) normal distribution, and are hence well-suited for use in training computer-implemented models. Thus, the predictive application 106 generates a difference feature in the training data 118. The difference feature is to be the target variable of the computer-implemented model 116. The predictive application 106 may also perform other transformations upon the training data 118. The predictive application 106 may also perform corresponding operations on the test data 120 in order to generate difference values for the test data 120.

The predictive application 106 selects the difference feature and at least one additional feature in the features in the training data 118. In an example, the predictive application may select each feature (and their corresponding values) in the training data 118. The predictive application 106 then trains the computer-implemented model 116 based upon the difference values and values for the at least one additional feature. The predictive application 106 may construct additional computer-implemented models (not depicted in FIG. 1), wherein parameters of each of the additional computer-implemented models vary from those of the computer-implemented model 116. For instance, features of the computer-implemented model 116 may include the difference feature, a first feature and a second feature, while features of a second computer-implemented model (not shown in FIG. 1) may include the difference features, the first feature, the second feature, and a third feature. The predictive application 106 (via the training module 108) may then perform hyperparameter tuning to identify a model that minimizes root mean square error (RMSE) through n-fold cross validation, where n is a positive integer greater than one. In an example, the computer-implemented model 116 minimizes the RMSE. The predictive application 106 tests performance of the computer-implemented model 116 using the test data 120.

Subsequently, the predictive application 106 receives clinical data for a patient that is to undergo treatment for the medical problem and a pre-treatment score value for the patient, the pre-treatment score value being indicative of a condition of the patient prior to undergoing the treatment. The pre-treatment score value has been generated based upon answers to questions in a questionnaire that have been provided by the patient (described above). The clinical data for the patient includes values that correspond to features upon which the computer-implemented model 116 was trained. However, the clinical data for the patient does not include a post-treatment score value or a difference value.

The predictive application 106 provides the values in the clinical data for the patient and the pre-treatment score value for the patient as input to the computer-implemented model 116. Based upon the input, the computer-implemented model 116, when executed by the predictive application 106, outputs a predicted difference value that is indicative of a predicted change in the condition of the patient from a first point in time to a second point in time, the first point in time occurring prior to the patient undergoing the treatment, the second point in time occurring subsequent to the patient undergoing the treatment (e.g., three months after undergoing the treatment). Based upon the pre-treatment score value for the patient and the predicted difference value output by the computer-implemented model 116, the predictive application 106 generates a predicted post-treatment score value for the patient that corresponds to the second point in time that is to occur (e.g., three months after undergoing the treatment). The predictive application 106 may cause the predicted post-treatment score value (or an indication thereof) to be presented on a display.

Subsequently, the predictive application 106 may receive an indication that comprises a post-treatment score value for the patient. The predictive application 106 may update the computer-implemented model 116 based upon the indication. For instance, the predictive application 106 may retrain the computer-implemented model 116 with an additional feature, may retrain the computer-implemented model 116 after removing a feature, or may retrain the computer-implemented model 116 using different model parameters. Additionally, the predictive application 106 may update the computer-implemented model 116 such that the post-treatment score is an input feature to computer-implemented model 116. Furthermore, the predictive application 106 may combine the clinical data for the patient, the pre-treatment score value for the patient, and the post-treatment score value for the patient and include the combined clinical data, pre-treatment score value, and post-treatment score value in the training data 118 or the test data 120.

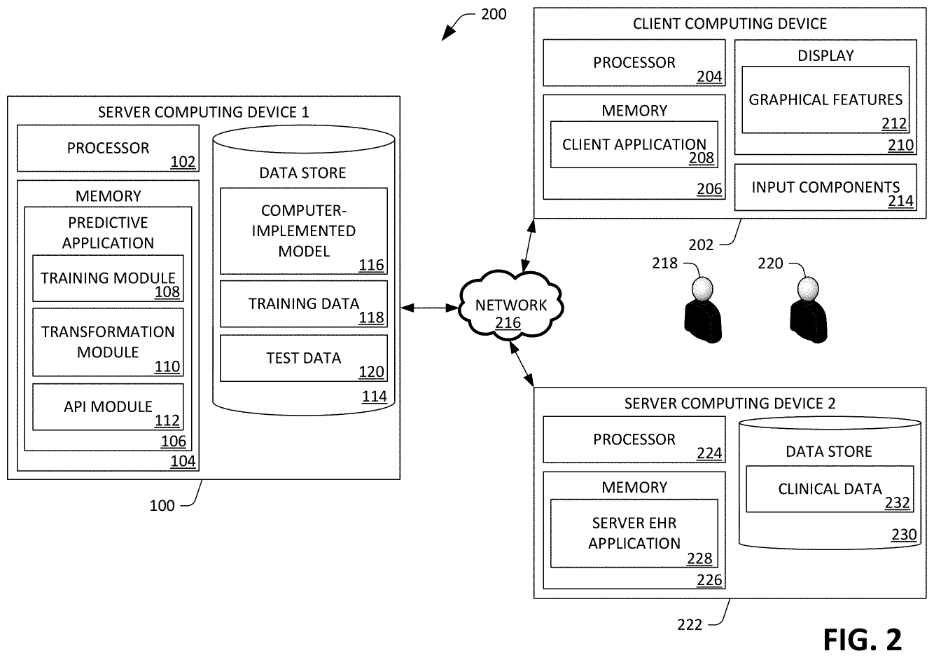

Referring now to FIG. 2, another exemplary computing system 200 that facilitates generating patient-specific outcome predictions is illustrated. The computing system 200 includes the computing system 100 (now referred to herein as “the first server computing device 100” for clarity) and its respective components described above.

The computing system 200 further includes a client computing device 202 that is in communication with the first server computing device 100 by way of a network 216 (e.g., the Internet, intranet, etc.). The client computing device 202 is operated by a healthcare worker 218 (e.g., a surgeon, a clinician, a nurse, etc.). The healthcare worker 218 may be providing care to a patient 220 at a healthcare facility. In an example, the client computing device 202 may be a desktop computing device, a laptop computing device, a tablet computing device, a smartphone, etc.

The client computing device 202 comprises a processor 204 and memory 206, wherein the memory 206 has a client application 208 loaded therein. In general, the client application 208, when executed by the processor 204, is configured to communicate with the predictive application 106 in order to cause a predicted post-treatment score value to be presented to the healthcare worker 218. In an embodiment, the client application 208 may be a web browser. In another embodiment, the client application 208 may be a client electronic health records (EHR) application (described below). In yet another embodiment, the client application 208 may be a standalone application that is configured to communicate with the predictive application 106.

The client computing device further comprises a display 210, whereupon graphical features 212 may be presented thereon. For instance, the graphical features 212 may include a graphical user interface (GUI) for the client application 208. The client computing device 202 also comprises input components 214 that enable the healthcare worker 218 to set forth input to the client computing device 202. For instance, the input components 214 may include a mouse, a keyboard, a trackpad, a scroll wheel, a microphone, a camera, a video camera, etc.

The computing system 200 may also include a second server computing device 222. The second server computing device 222 may be in communication with the first server computing device 100 and/or the client computing device 202 by way of the network 216 (or another network). The second server computing device 222 comprises a processor 224 and memory 226, wherein the memory 226 has a server EHR application 228 loaded therein. In general, the server EHR application 228, when executed by the processor 224, is configured to perform computer-executable tasks that facilitate treatment and care of patients in a healthcare facility (e.g., patient intake, electronic prescription generation, patient health record creation and maintenance, etc.). To this end, the server EHR application 228 is configured to communicate with a client EHR (e.g., the client application 208). The server computing device 222 further comprises a data store 230 that stores clinical data 232 for patients, wherein the server EHR application 228 is configured to maintain the clinical data 232 in the data store 230. The clinical data 232 may include electronic health records, prescription records, claims data, patient/disease registries, health surveys data, clinical trials data, etc.

Operation of the computing system 200 is now set forth. It is contemplated that the patient 220 is to undergo a treatment for a medical problem being experienced by the patient 220. It is further contemplated that the computer-implemented model 116 has been trained and tested as set forth above in the description of FIG. 1.

The client application 208 receives input from the healthcare worker 218 that causes the client application 208 to present a questionnaire on the display 210, the questionnaire comprising questions pertaining to the patient 220 and the medical problem being experienced by the patient 220. Alternatively, the patient 220 may interact with a virtual conversational assistant executing on the client computing device 202, and the virtual conversational assistant may identify the questionnaire based upon a conversation with the patient 220. In an example, the medical problem is a shoulder tear, and hence the questions in the questionnaire pertain to the shoulder tear of the patient 220. The healthcare worker 218 and the patient 220 review the questions on the display 210 and the client application 208 receives answers to the questions as input. The client application 208 generates a pre-treatment score value (e.g., an ASES) for the patient 220 based upon the answers.

The client application 208 then receives input from the healthcare worker 218 that causes an API call to be generated. In a first example, the client application 208 can transmit an identifier for the patient 220 to the server EHR application 228 responsive to receiving the input. Responsive to receiving the identifier for the patient 220, the server EHR application 228 executes a search over the clinical data 232 stored in the data store 230 based upon the identifier for the patient 220. The search produces search results that include clinical data for the patient 220. The server EHR application 228 transmits the clinical data for the patient 220 to the client application 208. Responsive to receiving the clinical data for the patient 220, the client application 208 constructs the API call, the API call comprising the pre-treatment score value for the patient 220 and the clinical data for the patient 220. The API call may also comprise an identifier for the patient 220, an identifier for the medical problem, and/or an identifier for the treatment for the medical problem. The client application 208 then transmits the API call to the predictive application 106.

In a second example, the client application 208 can transmit the pre-treatment score for the patient 220 and an identifier for the patient 220 to the server EHR application 228. The server EHR application 228 executes a search based upon the identifier for the patient 220 in order to retrieve the clinical data for the patient 220. The server EHR application 228 then constructs the API call (described above) and transmits the API call to the predictive application 106.

In a third example, the client application 208 receives the clinical data for the patient 220 as input from the healthcare worker 218. The client application 208 constructs the API call (described above) and transmits the API call to the predictive application 106.

Responsive to receiving the API call comprising the pre-treatment score value for the patient 220 and the clinical data for the patient 220, the predictive application 106 processes the API call. More specifically, the predictive application 106 provides the pre-treatment score value for the patient 220 and values in the clinical data for the patient 220 as input to the computer-implemented model 116. Based upon the input, the computer-implemented model 116, when executed by the predictive application 106, outputs a predicted difference value that is indicative of a predicted change in the condition of the patient 220 from a first point in time to a second point in time. Based upon the predicted difference value and the pre-treatment score value, the predictive application 106 generates a predicted post-treatment score value. Responsive to generating the predicted post-treatment score value, the predictive application 106 transmits the predicted post-treatment score value to the client application 208.

Responsive to receiving the predicted post-treatment score value from the predictive application 106, the client application 208 presents the predicted post-treatment score value on the display 210 as part of the graphical features 212. For instance, the predicted post-treatment score value may be presented within a GUI. In an embodiment, the predicted post-treatment score value may be presented in a plot of scores versus time (described in greater detail below). The client computing device 208 may cause the predicted post-treatment score to be stored in computer-readable storage (e.g., the data store 230). While the predictive application 106 has been described as generating a single predicted post-treatment score value corresponding to a single time after the patient 220 has undergone the treatment, it is to be understood the predicted application 220 may utilize the above-described processes to generate and store more than one predicted post-treatment score value for different periods of time. For instance, the predictive application 106, via execution of the computer-implemented model 116, may generate and store a three month predicted post-treatment score value, a six month predicted post-treatment score value, and a twelve month predicted post-treatment score value.

It is then contemplated that the patient undergoes the treatment for the medical problem and that an amount of time (e.g., three months, six months, twelve months, etc.) elapses from a time at which patient underwent the treatment. In an example, the patient 220 may visit the healthcare worker 218 for a follow-up appointment three months after the patient 220 has undergone the treatment for the medical problem.

The client application 208 receives input from the healthcare worker 218 that causes the client application 208 to present the questionnaire that was used to generate the pre-treatment score value on the display 210, the questionnaire comprising the questions pertaining to the patient 220 and the medical problem of the patient 220. The healthcare worker 218 and the patient 220 review the questions in the questionnaire and the client application 208 receives second answers to the question as input, the second answers being provided by the patient 220. As the patient 220 has already undergone the treatment, the second answers likely vary from the answers to the questions provided prior to the patient 220 undergoing the treatment. The client application 208 generates a post-treatment score value (e.g., a second ASES) for the patient 220 based upon the second answers.

The client application 208 may then receive an identifier for the patient 220 as input. Responsive to receiving the identifier for the patient 220, the client application 208 retrieves the predicted post-treatment score value(s) from the computer-readable storage, and presents the predicted post-treatment score value(s) and the post-treatment score value (or graphical data that is indicative of the predicted post-treatment score value(s) and the post-treatment score value) on the display 210 as part of the graphical features 212.

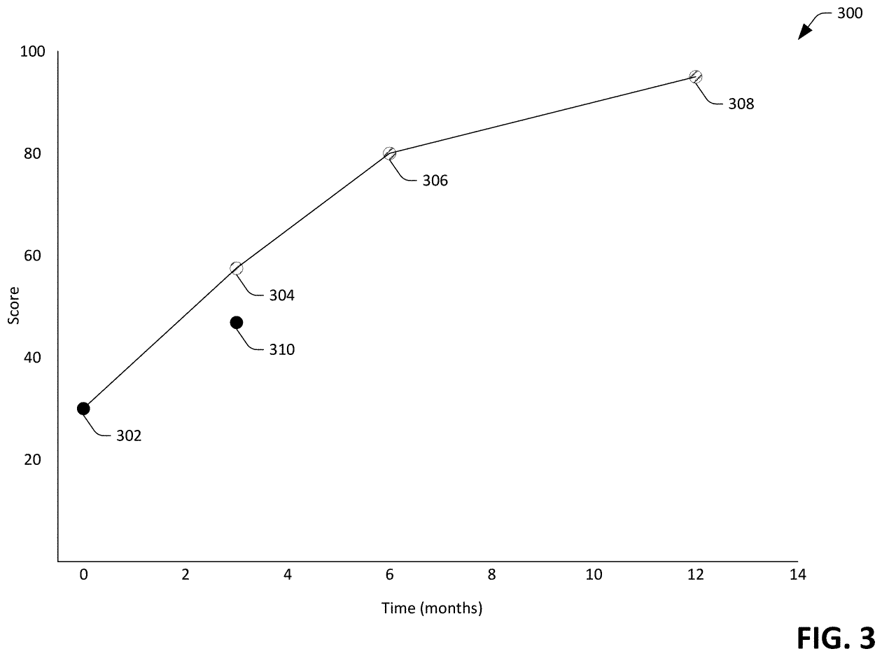

Turning now to FIG. 3, an exemplary plot 300 of scores (y-axis) versus time (x-axis) is illustrated. The predictive application 106 may cause the plot 300 to be presented on the display 210 (e.g., within a GUI for the client application 208). The plot 300 comprises a first graphical indicator 302 that is indicative of the pre-treatment score value, a second graphical indicator 304 that is indicative of the three month predicted post-treatment score value, a third graphical indicator 306 that is indicative of the six month predicted post-treatment score value, and a fourth graphical indicator 308 that is indicative of the twelve month predicted post-treatment score value. The plot 300 also includes a fifth graphical indicator 310 that is indicative of the three month post-treatment score value that was generated by the client application 208 during the three-month appointment between the healthcare worker 218 and the patient 220.

The healthcare worker 218 may review the plot 300 to determine whether the treatment has been effective in treating the medical problem of the patient 220. For instance, as the three month post-treatment score value (indicated by the fifth graphical indicator 310) is less than the three month predicted post-treatment score value (indicated by the second graphical indicator 304), the healthcare worker 218 may conclude that the patient 220 is not recovering as expected. The healthcare worker 220 may then tailor further treatment for the patient 220 such that the patient 220 recovers at an expected rate. In an embodiment, the healthcare worker 218 may utilize the plot 300 and additional clinical decision support services provided by the server EHR application 228 (or other applications, not shown in FIG. 2) in order to make further treatment decisions for the medical problem of the patient 220.

The client application 208 may transmit the three month post-treatment score value to the predictive application 106. The predictive application 106 may update the computer-implemented model 116 based upon the three month post-treatment score value (described above in the description of FIG. 1).

Although the computing system/first server computing device 100 has been described above as training a single computer-implemented model for a single treatment for a single medical problem, it is to be understood that the computing system/first server computing device 100 may train many different computer implemented models for many different treatments for different medical problems.

For instance, for the medical problem that the patient 220 is experiencing, there may be a first treatment (e.g., a first surgical implant, a first type of surgery, a first medication, a first surgeon that performs a surgery, etc.) and a second treatment (e.g., a second surgical implant, a second type of surgery, a second medication, a second surgeon that performs the surgery, etc.) available, and the healthcare worker 218 may wish to ascertain which treatment should be pursued. As such, the training data 118 may comprise a first subset corresponding to patients that underwent the first treatment for the medical problem and a second subset corresponding to patients that underwent the second treatment for the medical problem. Using the above-described processes, the predictive application 106 may generate a first computer-implemented model assigned to the first treatment and a second computer-implemented model assigned to the second treatment based upon the first subset of training data 118 and the second subset of training data 118, respectively. Prior to the healthcare worker 218 and the patient 220 deciding upon whether the patient 220 should undergo the first treatment or the second treatment for the medical problem, the predictive application 106 may provide values in the clinical data for the patient 220 and the pre-treatment score value as input to each of the first computer-implemented model and the second computer-implemented model. Based upon the input, the first computer-implemented model and the second computer-implemented model output a first predicted difference value and a second predicted difference value, respectively. The predictive application 106 generates a first predicted post-treatment score value for the patient 220 based upon the pre-treatment score value and the first predicted difference value. The predictive application 106 also generates a second predicted post-treatment score value for the patient 220 based upon the pre-treatment score value and the second predicted difference value. The predictive application 106 may cause the first predicted post-treatment score value and the second predicted post-treatment score value to be presented to the healthcare worker 218 on the display 210. The healthcare worker 218 may then recommend either the first treatment or the second treatment based upon a comparison between the first predicted post-treatment score value and the second predicted post-treatment score value. Alternatively, in an embodiment, factors pertaining to each of the first treatment and the second treatment may be used as features of the computer-implemented model 116, and the computer-implemented model 116 may be configured to output a value which may be used to determine whether the first treatment or the second treatment is preferable for the patient 220. Speaking more generally, the computer-implemented model is able to pick and recommend the best treatment out of multiple treatment options.

While the above-described technologies have been described above outputting a predicted post-treatment score value based upon a pre-treatment score value and a predicted difference value, other possibilities are contemplated. For instance, the above-described technologies may be utilized in situations after the patient 220 has undergone the treatment. In such a case, the input to the computer-implemented model 116 may be a score value for the patient 220 that has been mostly recent reported (e.g., a score value taken three months after the patient 220 undergoing the treatment), as opposed to a pre-treatment score value. Likewise, the target variable of the computer-implemented model 116 may be a difference between a mostly recently reported score (e.g., a score taken at three months after the treatment) and another score (e.g., a score taken at six months after the treatment). Output of the computer-implemented model 116 may then be a predicted difference value that is indicative of a predicted change in the condition of a patient from a first point in time (e.g., three months after undergoing the treatment) to a second point in time (e.g., six months after undergoing the treatment).

Although a difference between a post-treatment score and a pre-treatment score has been described above as being the target variable for the computer-implemented model 116, it is to be understood that other possibilities are contemplated for the target variable. For instance, the predictive application 106 may perform a transformation (e.g., a log transformation, an exponential transformation, a square transformation, a cubic transformation, a square root transformation, a cubic root transformation, a power transformation, a Box-Cox transformation, a Yeo-Johnson transformation, etc.) on score values (e.g., pre-treatment score values, post-treatment scores values, and/or a difference between post-treatment score values and pre-treatment score values) in order to generate a transformed score feature and corresponding values for the transformed score feature. The transformation may transform a distribution of the score values from (approximately) skew to (approximately) normal. The predictive application 106 may then utilize the transformed feature as the target variable for the computer-implemented model 116. It is also to be understood the predictive application 106 may perform an inverse transformation on a predicted score value output by the computer-implemented model in order to present the predicted score value in a manner that is readily understood by the healthcare worker 218 and/or the patient 220.

Although the predictive application 106 has been described above as being primarily utilized in the context of an interaction between the healthcare worker 218 and the patient 220, other possibilities are contemplated. For instance, in an embodiment, the predictive application 106 may be an application that runs on a mobile computing device of the patient 220. Similar to the processes described above, the predictive application 106 may present a questionnaire to the patient 220 (e.g., through a virtual conversational assistant), and the predictive application 106 may receive answers to questions in the questionnaire as input from the patient 220, and the predictive application 106 may output an indication, in real-time, of a predicted score value at some time in the future. The patient 220 may modify his/her behavior based upon the predicted score value. For instance, if the predicted score value is below a certain target score value, the patient 220 may increase his/her exercise habits, diet, etc. Thus, it is to be understood that the predictive application 106 may function outside of the healthcare worker 218/patient 220 relationship.

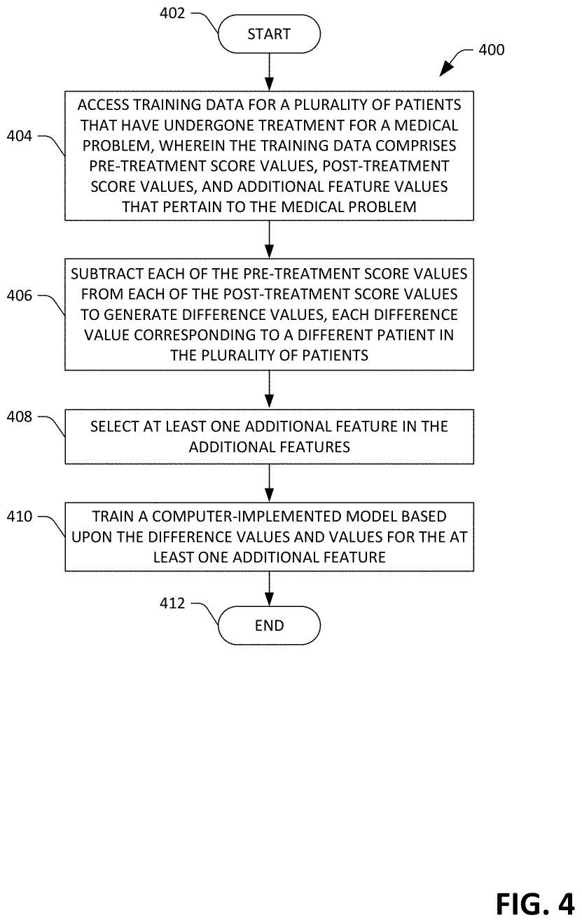

FIGS. 4-6 illustrate exemplary methodologies relating to generating patient-specific outcome predictions. While the methodologies are shown and described as being a series of acts that are performed in a sequence, it is to be understood and appreciated that the methodologies are not limited by the order of the sequence. For example, some acts can occur in a different order than what is described herein. In addition, an act can occur concurrently with another act. Further, in some instances, not all acts may be required to implement a methodology described herein.

Moreover, the acts described herein may be computer-executable instructions that can be implemented by one or more processors and/or stored on a computer-readable medium or media. The computer-executable instructions can include a routine, a sub-routine, programs, a thread of execution, and/or the like. Still further, results of acts of the methodologies can be stored in a computer-readable medium, displayed on a display device, and/or the like.

Referring now to FIG. 4, a methodology 400 performed by a computing system that facilitates training a computer-implemented model that generates patient-specific outcome predictions is illustrated. The methodology 400 begins at 402, and at 404, the computing system accesses training data. The training data comprises values for features that are associated with a plurality of patients that have undergone a treatment for a medical problem. The features comprise a pre-treatment score, a post-treatment score, and additional features comprising factors pertaining to the medical problem. Likewise, the values for the features comprise pre-treatment score values, post-treatment score values, and additional feature values that pertain to the medical problem as experienced by each of the plurality of patients. At 406, the computing system subtracts each of the pre-treatment score values from each of the post-treatment score values to generate difference values (and hence, a difference feature), each difference value in the difference values corresponding to a different patient in the plurality of patients. At 408, the computing system selects at least one additional feature in the additional features. At 410, the computing system trains the computer-implemented model based upon the difference values and values for the at least one additional feature. The computer-implemented model is configured to output, based upon input values, a predicted difference value that is indicative of a change in a condition of a patient from a first point in time to a second point in time, the first point in time occurring prior to the patient undergoing the treatment, the second point in time occurring subsequent to the patient undergoing the treatment. The methodology 400 concludes at 412.

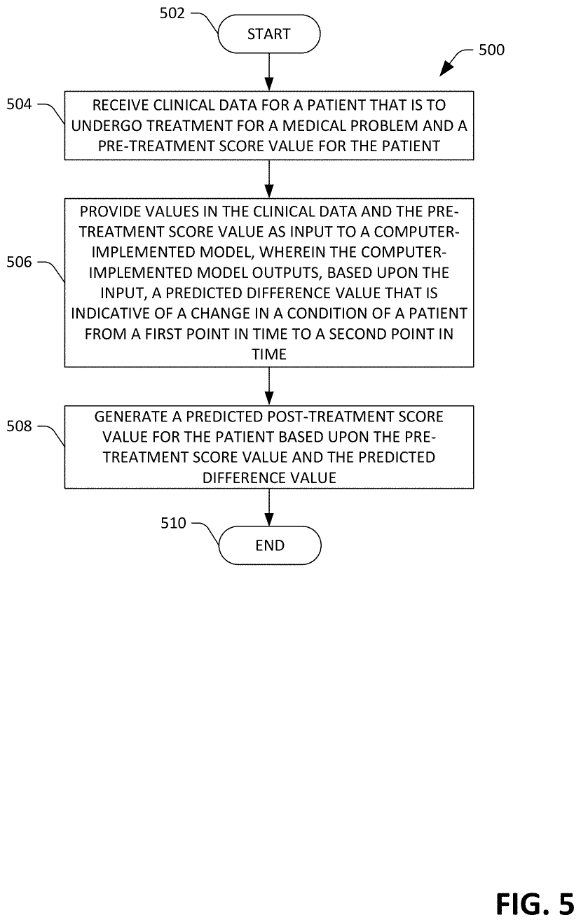

Turning now to FIG. 5, a methodology 500 performed by a computing system that facilitates executing a computer-implemented model that generates patient-specific outcome predictions is illustrated. The methodology 500 begins at 502, and at 504, the computing system receives clinical data for a patient that is to undergo a treatment for a medical problem and a pre-treatment score value for the patient. The pre-treatment score value is indicative of a condition of the patient prior to the patient undergoing the treatment. At 506, the computing system provides values in the clinical data and the pre-treatment score value as input to a computer-implemented model. The computer-implemented model has been trained using training data comprising values for features associated with a plurality of patients that have undergone the treatment. A feature in the features comprises a difference between a post-treatment score and a pre-treatment score for the treatment. The computer-implemented model outputs, based upon the values in the clinical data and the pre-treatment score value for the patient, a predicted difference value that is indicative of a predicted change in the condition of the patient from a first point in time to a second point in time, the first point in time occurring prior to the patient undergoing the treatment, the second point in time occurring subsequent to the patient undergoing the treatment. At 508, the computing system generates a predicted post-treatment score value for the patient based upon the pre-treatment score value and the predicted difference value. The predicted post-treatment score value for the patient is presented on a display of a computing device. The methodology 500 concludes at 510.

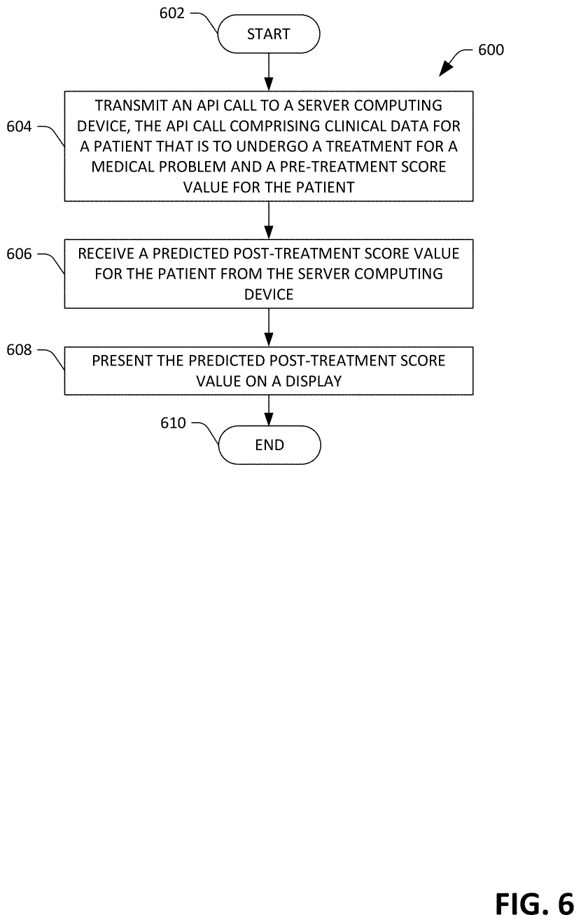

With reference now to FIG. 6, a methodology 600 performed by a client computing device that facilitates generating patient-specific outcome predictions is illustrated. The methodology 600 begins at 602, and at 604, the client computing device transmits, over a network connection, an API call to a server computing device. The API call comprises clinical data for a patient and a pre-treatment score value for the patient, the pre-treatment score value for the patient being indicative of a condition of the patient prior to undergoing treatment for a medical problem. Responsive to receiving the clinical data for the patient and the pre-treatment score value, the server computing device provides values in the clinical data and the pre-treatment score value to a computer-implemented model as input. The computer-implemented model has been trained using training data comprising values for features associated with a plurality of patients that have undergone the treatment. A feature in the features comprises a difference between a post-treatment score and a pre-treatment score. The computer-implemented model outputs, based upon the input, a predicted difference value that is indicative of a predicted change in the condition of the patient from a first point in time to a second point in time, the first point in time occurring prior to the patient undergoing the treatment, the second point in time occurring subsequent to the patient undergoing the treatment. The computing system generates a predicted post-treatment score value for the patient based upon the pre-treatment score value and the predicted difference value. At 606, the client computing device receives, over the network connection, the predicted post-treatment score value for the patient. At 608, responsive to receiving the predicted post-treatment score value for the patient, the client computing device presents the predicted post-treatment score value on a display. The methodology 600 concludes at 610.

### EXAMPLES

To demonstrate the benefits of the above-described technologies, an experiment was performed. Training data was obtained from a global registry that comprises de-identified patient data. The training data included data for patients that underwent shoulder arthroscopy surgery between 2011 and 2020 and that completed pre-surgery ASES questionnaires, three month post-surgery ASES questionnaires, six month post-surgery ASES questionnaires, and twelve month post-surgery ASES questionnaires. Thus, the training data included pre-surgery ASESs, three month post-surgery ASESs, six month post-surgery ASESs, and twelve month post-surgery ASESs. Patient demographic information and procedure-related information such as gender, age, BMI, smoker, diabetic, tendons torn, tendon quality, cofield tear size, retraction stage, tear shape, medial anchor type, medial knotless anchors, medial suture anchors, lateral anchor type, lateral knotless anchors, lateral suture anchors, PT VAPS, PT score, and year of shoulder arthroscopy procedure were utilized as predictors.

The training data included data for 631 patients. The 631 patients were between 24-83 years old at the time of the surgery and had all of the aforementioned predictors. The 631 patients included 362 males (approximately 57%) and 269 females (approximately 43%). The mean age of the 631 patients was 61.5 years.

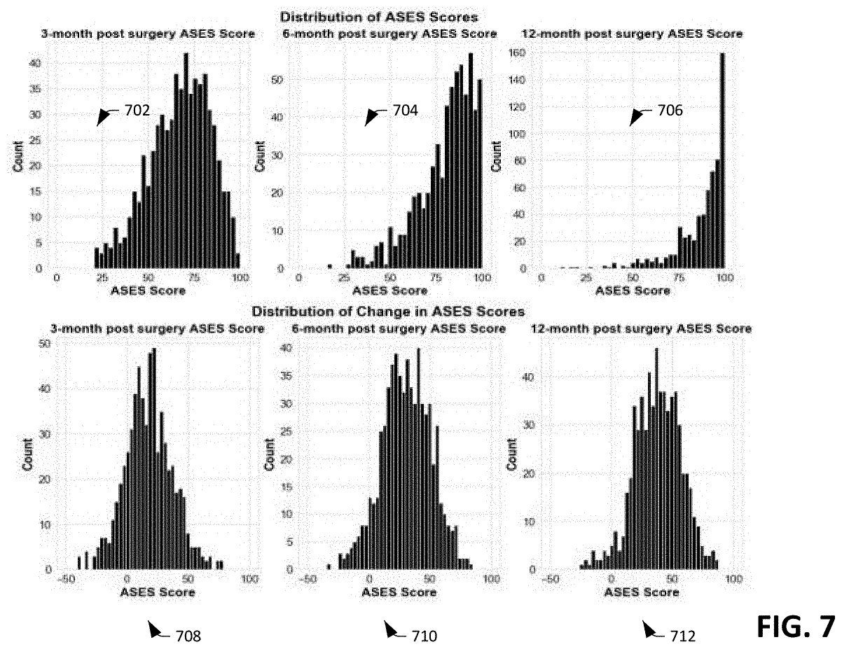

With reference now to FIG. 7, histograms 702-712 for the 631 patients included in the experiment are illustrated. The x-axis for each histogram in the histograms 702-712 represents ASES and the y-axis for each histogram in the histograms 702-712 represents a number of patients. Histograms 702, 704, and 706 represent ASES at three months after surgery, six months after surgery, and twelve months after surgery, respectively. As shown in FIG. 7, the distributions of ASESs reflected in histograms 702-706 are skewed, particular with respect to histogram 706, which shows that twelve months after surgery, most patients have an ASES above 80.

However, a computer-implemented model that predicts a target variable that has a normal distribution tends to generate more accurate predictions than a computer-implemented model that predicts a target variable that has a skewed distribution. As such, a difference between the post-surgery ASES and the pre-surgery ASES for each of the 631 patients was taken. Histogram 708 represents a difference between the three month post-surgery ASES and the pre-surgery ASES for each patient, histogram 710 represents a difference between the six month post-surgery ASES and the pre-surgery ASES for each patient, and histogram 712 represents a difference between the twelve month post-surgery ASES and the pre-surgery ASES for each patient. Based on comparisons between histogram 702 and histogram 708, histogram 704 and histogram 710, and histogram 706 and histogram 712, the difference between the post-surgery ASES and the pre-surgery ASES is more normally distributed than that of the distribution of the post-surgery ASES alone. Thus, the difference was utilized as the target variable in the computer-implemented model.

The training data was randomly split into a training set (80%) and a test set (20%). Computer-implemented regression models were generated using the training set. Hyperparameter tuning was performed to identify the most accurate computer-implemented model based on minimizing root mean squared error (RMSE) through 5-fold cross-validation. Performance of the highest performing computer-implemented model was evaluated on the test set to gauge performance.

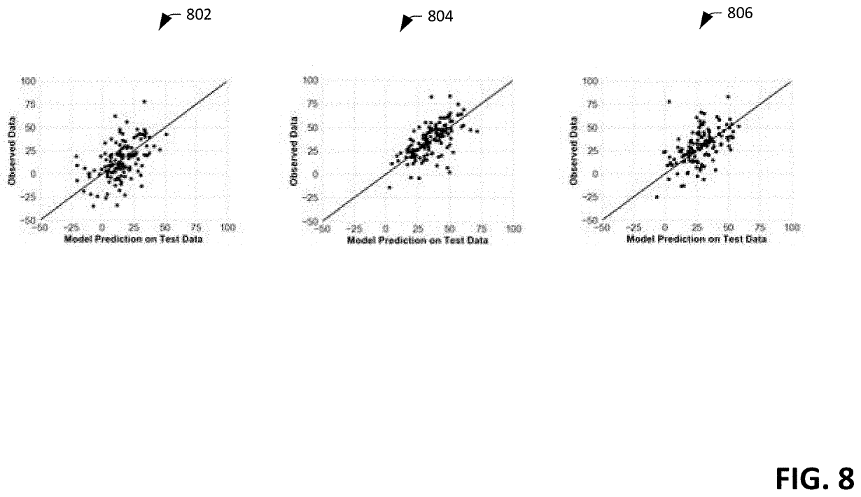

Turning now to FIG. 8, parity plots 802-806 are illustrated. Plot 802 corresponds to a predicted change in ASES for three months after surgery, plot 804 corresponds to a predicted change in ASES for six months after surgery, and plot 806 corresponds to a predicted change in ASES for twelve months after surgery. The coefficient of determination (R²) for the three month, six month, and twelve month periods were 0.20, 0.22, and 0.36, respectively. The computer-implemented model was able to predict overall trends in patient recovery in many cases. The computer-implemented model may be used conjunction with other clinical decision support tools to enable surgeons and other healthcare workers to provide more customized care for patients. Furthermore, the computer-implemented model can help to identify high-risk patients early on such that additional care may be provided to such patients.

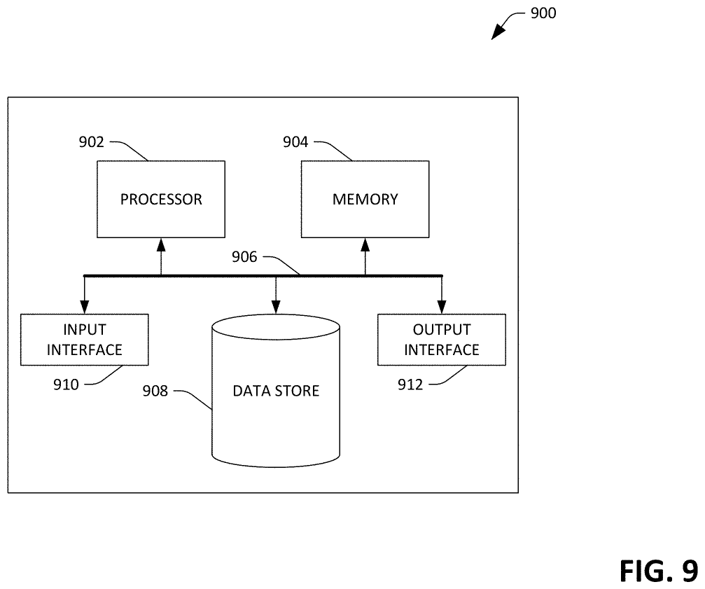

Referring now to FIG. 9, a high-level illustration of an exemplary computing device 900 that can be used in accordance with the systems and methodologies disclosed herein is illustrated. For instance, the computing device 900 may be used in a system that trains a computer-implemented model that generates patient-specific outcome predictions when executed. By way of another example, the computing device 900 can be used in a system that executes a computer-implemented model that generates patient-specific outcome predictions. Thus, the computing device 900 may be or include the computing system 100 (also referred to herein as the first server computing device 100), the second server computing device 222, and/or the client computing device 202 and the computing system 100 (also referred to herein as the first server computing device 100), the second server computing device 222, and/or the client computing device 202 may be or include the computing device 900. The computing device 900 includes at least one processor 902 that executes instructions that are stored in a memory 904. In an example, the at least processor 902 may be a central processing unit (CPU) or a graphics processing unit (GPU). The instructions may be, for instance, instructions for implementing functionality described as being carried out by one or more components discussed above or instructions for implementing one or more of the methods described above. The processor 902 may access the memory 904 by way of a system bus 906. In addition to storing executable instructions, the memory 904 may also store computer-implemented models, training data, test data, clinical data for patients, questionnaires, answers to questions in the questionnaires, etc.

The computing device 900 additionally includes a data store 908 that is accessible by the processor 902 by way of the system bus 906. The data store 908 may include executable instructions, computer-implemented models, training data, test data, clinical data for patients, questionnaires, answers to questions in the questionnaires, etc. The computing device 900 also includes an input interface 910 that allows external devices to communicate with the computing device 900. For instance, the input interface 910 may be used to receive instructions from an external computer device, from a user, etc. The computing device 900 also includes an output interface 912 that interfaces the computing device 900 with one or more external devices. For example, the computing device 900 may display text, images, etc. by way of the output interface 912.

It is contemplated that the external devices that communicate with the computing device 900 via the input interface 910 and the output interface 912 can be included in an environment that provides substantially any type of user interface with which a user can interact. Examples of user interface types include graphical user interfaces, natural user interfaces, and so forth. For instance, a graphical user interface may accept input from a user employing input device(s) such as a keyboard, mouse, remote control, or the like and provide output on an output device such as a display. Further, a natural user interface may enable a user to interact with the computing device 900 in a manner free from constraints imposed by input devices such as keyboards, mice, remote controls, and the like. Rather, a natural user interface can rely on speech recognition, touch and stylus recognition, gesture recognition both on screen and adjacent to the screen, air gestures, head and eye tracking, voice and speech, vision, touch, gestures, machine intelligence, and so forth.

Additionally, while illustrated as a single system, it is to be understood that the computing device 900 may be a distributed system. Thus, for instance, several devices may be in communication by way of a network connection and may collectively perform tasks described as being performed by the computing device 900.

Various functions described herein can be implemented in hardware, software, or any combination thereof. If implemented in software, the functions can be stored on or transmitted over as one or more instructions or code on a computer-readable medium. Computer-readable media includes computer-readable storage media. A computer-readable storage media can be any available storage media that can be accessed by a computer. By way of example, and not limitation, such computer-readable storage media can comprise random-access memory (RAM), read-only memory (ROM), electrically erasable programmable read-only memory (EEPROM), compact disc read-only memory (CD-ROM) or other optical disk storage, magnetic disk storage or other magnetic storage devices, or any other medium that can be used to carry or store desired program code in the form of instructions or data structures and that can be accessed by a computer. Disk and disc, as used herein, include compact disc (CD), laser disc, optical disc, digital versatile disc (DVD), floppy disk, and blu-ray disc (BD), where disks usually reproduce data magnetically and discs usually reproduce data optically with lasers. Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio and microwave are included in the definition of communication medium. Combinations of the above should also be included within the scope of computer-readable media.

Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim.

What is claimed is:

1. A computing system, comprising:
a processor; and
memory storing instructions that, when executed by the processor, cause the processor to perform acts comprising:
receiving clinical data for a patient that is to undergo a treatment for a medical problem and a pre-treatment score value for the patient that is indicative of a condition of the patient prior to the patient undergoing the treatment;
providing values in the clinical data and the pre-treatment score value as input to a computer-implemented model, wherein the computer-implemented model has been trained using training data comprising values for features associated with a plurality of patients that have undergone the treatment for the medical problem, wherein a feature in the features comprises a difference between a post-treatment score and a pre-treatment score for the treatment, wherein the computer-implemented model outputs, based upon the values in the clinical data and the pre-treatment score value for the patient, a predicted difference value that is indicative of a predicted change in the condition of the patient from a first point in time to a second point in time, the first point in time occurring prior to the patient undergoing the treatment, the second point in time occurring subsequent to the patient undergoing the treatment; and
based upon the pre-treatment score value for the patient and the predicted difference value, generating a predicted post-treatment score value for the patient, wherein an indication of the predicted post-treatment score value for the patient is presented on a display.

2. The computing system of claim 1, wherein the treatment for the medical problem is a surgical procedure.

3. The computing system of claim 2, wherein the medical problem is a shoulder injury, wherein the surgical procedure is a shoulder arthroscopy.

4. The computing system of claim 1, wherein a second feature in the features comprises a factor pertaining to the medical problem.

5. The computing system of claim 1, wherein the pre-treatment score and the post-treatment score are American Shoulder and Elbow Surgeons Scores (ASESs)

6. The computing system of claim 1, the acts further comprising:
prior to providing the values in the clinical data and the pre-treatment score value as input to the computer-implemented model, training the computer-implemented model based upon the training data.

7. The computing system of claim 6, wherein the values for the features comprise post-treatment score values for the plurality of patients and pre-treatment score values for the plurality of patients, the acts further comprising:
prior to training the computer-implemented model, subtracting each of the pre-treatment score values from each of the post-treatment score values to generate difference values for the plurality of patients, each difference value in the difference values corresponds to a different patient in the plurality of patients, wherein the computer-implemented model is trained in part upon the difference values.

8. The computing system of claim 7, wherein the post-treatment score values for the plurality of patients have a skewed distribution, wherein the difference values for the plurality of patients have a normal distribution.

9. The computing system of claim 1, wherein the acts are performed by a module of an electronic health records (EHR) application.

10. The computing system of claim 1, the acts further comprising:
subsequent to generating the predicted post-treatment score value for the patient, receiving an indication that comprises a post-treatment score value for the patient that has been generated subsequent to the patient undergoing the treatment for the medical problem;
updating the computer-implemented model based upon the indication; and
generating a second treatment for the medical problem based upon the updated computer-implemented model.

11. The computing system of claim 1, wherein the computer-implemented model is at least one of: a gradient boosted decision tree, a linear regression, a lasso, a ridge regression, a decision tree, an artificial neural network, a support vector machine, a hidden Markov model, a recurrent neural network, a deep neural network, or a convolutional neural network.

12. A method executed by a processor of a computing system, comprising:
receiving clinical data for a patient that is to undergo a treatment for a medical problem and a pre-treatment score value for the patient that is indicative of a condition of the patient prior to the patient undergoing the treatment;
providing values in the clinical data and the pre-treatment score value as input to a computer-implemented model, wherein the computer-implemented model has been trained using training data comprising values for features associated with a plurality of patients that have undergone the treatment, wherein a feature in the features comprises a difference between a post-treatment score and a pre-treatment score for the treatment, wherein the computer-implemented model outputs, based upon the values in the clinical data and the pre-treatment score value for the patient, a predicted difference value that is indicative of a predicted change in the condition of the patient from a first point in time to a second point in time, the first point in time occurring prior to the patient undergoing the treatment, the second point in time occurring subsequent to the patient undergoing the treatment; and
based upon the pre-treatment score value for the patient and the predicted difference value, generating a predicted post-treatment score value for the patient, wherein an indication of the predicted post-treatment score value for the patient is presented on a display of a computing device.

13. The method of claim 12, further comprising:
prior to providing the values in the clinical data and the pre-treatment score value for the patient as input to the computer-implemented model, training the computer-implemented model based upon the training data.

14. The method of claim 13, wherein the values for the features comprise a first post-treatment score value for a first patient in the plurality of patients and a first pre-treatment score value for the first patient in the plurality of patients, the method further comprising:
prior to training the computer-implemented model based upon the training data, subtracting the first pre-treatment score value from the first post-treatment score value to generate a first difference value, wherein the computer-implemented model is trained in part upon the first difference value.

15. The method of claim 1, wherein the computing system exposes an application programing interface (API) to the computing device, wherein the clinical data for the patient and the pre-treatment score value for the patient are received by the computing system as part of an API call generated by the computing device, wherein the computing system provides the values in the clinical data and the pre-treatment score value for the patient as input to the computer-implemented model responsive to the API call being received, wherein the computing system transmits the predicted post-treatment score value for the patient to the computing device responsive to generating the predicted post-treatment score value.

16. The method of claim 12, wherein the post-treatment score and the pre-treatment score are patient reported outcome measures (PROMs).

17. The method of claim 12, wherein values for the post-treatment score and values for the pre-treatment score for the treatment have been generated based upon answers to questions in a questionnaire pertaining to the medical problem, wherein the plurality of patients have provided the answers.

18. The method of claim 12, wherein the computer-implemented model is a regression model.

19. The method of claim 12, wherein the computing system receives the clinical data for the patient from an electronic health records (EHR) application.

20. A non-transitory computer-readable medium comprising instructions that, when executed by a processor of a server computing device, cause the processor to perform acts comprising:
receiving, from a client computing device, clinical data for a patient that has undergone a treatment for a medical problem and a score value for the patient that has been mostly reported, the score value being indicative of a condition of the patient at a first point in time after undergoing the treatment;
providing values in the clinical data and the score value as input to a computer-implemented model, wherein the computer-implemented model has been trained using training data comprising values for features associated with a plurality of patients that have undergone the treatment, wherein the features include a transformed score feature, wherein values for the transformed score features have been generated by applying a transformation on values for a score feature, wherein the values for the transformed score feature have a normal distribution, wherein the computer-implemented model outputs, based upon the values in the clinical data and the score value for the patient, a predicted score value corresponding to the transformed score feature that is indicative of a condition of at a second point in time, the second point in time occurring subsequent to the first point in time; and
transmitting the predicted score value for the patient to the client computing device, wherein the predicted score value for the patient is presented on a display of the client computing device.
